(19) United States
(12) Patent Application Publication
(10) Pub. No.: US 2026/0279533 A1
(43) Pub. Date: Sep. 17, 2026

(54) PATIENT ADHERENCE TO CARE PLANS

(71) Applicants: Brajendra S. Thakur, Pittsburgh, PA (US); Nancy Hunsberger, Mount Laurel, NJ (US); Nicole Blohm, Mount Pleasant, SC (US); Stephen Cousins, Nashville, TN (US)

(72) Inventors: Brajendra S. Thakur, Pittsburgh, PA (US); Nancy Hunsberger, Mount Laurel, NJ (US); Nicole Blohm, Mount Pleasant, SC (US); Stephen Cousins, Nashville, TN (US)

(73) Assignee: Ricoh Company, Ltd., Tokyo (JP)

(21) Appl. No.: 19/078,985

(22) Filed: Mar. 13, 2025

(51) Int. Cl. G16H 20/10; G06T 11/00; G06V 20/20; G16H 40/20; G16H 70/20

(52) U.S. Cl. CPC G16H 20/10; G06T 11/00; G06V 20/20; G16H 40/20; G16H 70/20; G06T 2210/41

(57) ABSTRACT

The present disclosure relates to improving patient adherence to care plans. Information associated with one or more medications may be received from a public database. An image of the one or more medications may be received. Dosage instructions for the one or more medications may be received from one or more medical provider devices. A dosage plan may be generated based at least in part on the information associated with the one or more medications, the image of the one or more medications, and the dosage instructions, A machine-readable code for accessing the dosage plan may be generated and provided to a client device.

### TECHNICAL FIELD

The present disclosure relates medical computer systems and, more particularly, to medical computer systems to assist patients in adhering to prescribed medical treatments.

### BACKGROUND

Medical patients with reduced cognitive or physical capacities often struggle to adhere to medication, food, and care plans. This non-adherence can include, for example, not taking medications on time or regularly, not taking the correct quantity of medications, not following dietary guidelines necessitated by their medications or care plans (e.g., take a medication before food, take a medication with water, refrain from ingesting alcohol or caffeine, etc.), and not following activity guidelines necessitated by their care plans (e.g., avoid driving after taking a medication, practice stretching exercises daily, etc.). Care plan non-adherence can be caused by simply forgetting to take a medication; confusion about how to correctly follow the care plan, like what medications need to be taken, when each medication needs to be taken, dosage of each medication, etc. ; fear of adverse reactions to medications; mental health factors that interfere with following the care plan; hesitancy in taking the medication due to mistrust, doubting the medication's necessity, or cost; difficulty in administering medication, such as inability to open a bottle or deliver an injection or difficulty with reading a medication label; difficulty in preparing or ingesting food required by the care plan; difficulty in refraining from activities restricted by the care plan or performing activities required by the care plan; or impediments to obtaining a timely supply of medications, food, or other supplies.

But ensuring care plan adherence from such patients is often difficult, especially when a caregiver does not live full-time in the patient's residence. Non-adherence with care plans can result in further medical complications, doctor and hospital visits, expenses, and impacts on family members. Complications resulting from non-adherence can lead to further difficulties and costs to ensure that those complications do not recur.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

In the drawings:

FIG. 1 illustrates a block diagram depicting an example of a network environment, in an embodiment.

FIGS. 2A-2D depict examples in which the AR interface is used to identify a medication related to a dosing event, in various embodiments.

FIG. 3 shows an example in which the AR interface is used to present a dosage plan, in an embodiment.

FIGS. 4A-4B depict examples in which the AR interface is used to prompt the patient to ensure completion of a current dosing event, in various embodiments.

FIG. 5 is a block diagram that illustrates a computer system upon which an embodiment of the invention may be implemented.

FIG. 6 is a block diagram of a basic software system that may be employed for controlling the operation of a computing system.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

### I. General Overview

The present disclosure relates to improving patient adherence to care plans. A summarization module may obtain data from various sources. The data may relate to one or more medications prescribed to a patient and other medical information of the patient. The summarization module may generate a dosage plan based on the data from the various sources. The dosage plan may instruct the patient on taking one or more medications.

The patient's mobile device, such as a smartphone or tablet, may be configured to present an augmented reality interface that assists the patient in adhering to the care plan. To assist with medication adherence, the augmented reality (AR) interface may, for instance, help the patient to correctly identify medications. The AR interface may further present dosage plans generated by the summarization module. A chatbot may interact with the patient to help ensure the patient completes dosage of one or more medications.

### II. System Overview

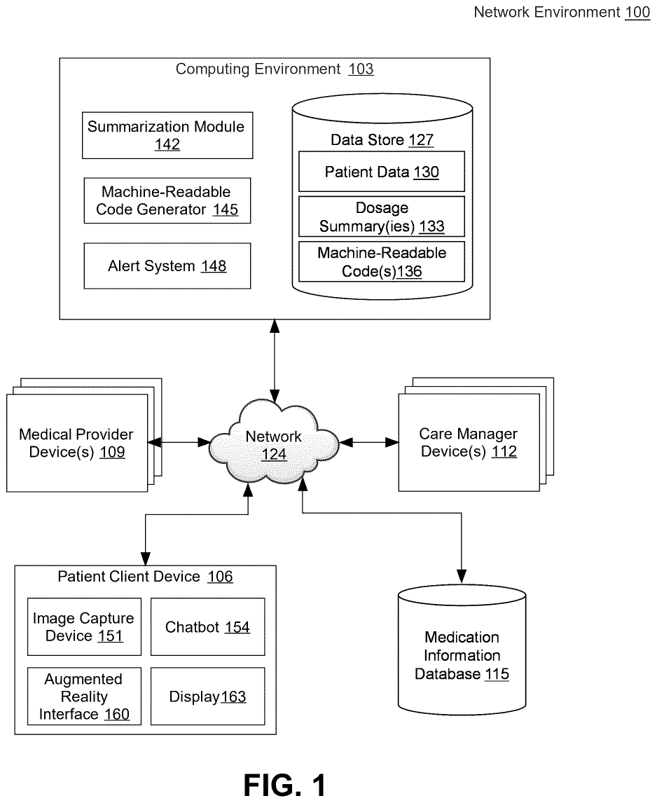

FIG. 1 illustrates a block diagram depicting an example of a network environment 100, in an embodiment. The network environment 100 may include a computing environment 103, a patient device 106, one or more medical provider devices 109, one or more care manager devices 112, a medication database 115, and potentially other components in communication via a network 124.

The network 124 includes the Internet, intranets, extranets, wide area networks (WANs), local area networks (LANs), wired networks, wireless networks, other suitable networks, or any combination of two or more such networks. The networks may include satellite networks, cable networks, Ethernet networks, and other types of networks.

The various components of the network environment 100 may interact via the network 124 using application programming interface(s)

### A. Computing Environment

The computing environment 103 may include a computing device, such as a server computer, that provides computing capabilities. Alternatively, the computing environment 103 may employ multiple computing devices that are arranged in one or more server banks or computer banks. In one example, the computing devices may be located in a single installation. In another example, the computing devices for the computing environment 103 may be distributed among multiple different geographical locations. In one case, the computing environment 103 may include multiple computing devices that together may form a hosted computing resource or a grid computing resource. In addition, the computing environment 103 may operate as an elastic computing resource where the allotted capacity of computing-related resources, such as processing resources, network resources, and storage resources, may vary over time. In other examples, the computing environment 103 may include or be operated as one or more virtualized computer instances that may be executed to perform the functionality that is described herein.

Various data may be stored in a data store 127 that may be accessible to the computing environment 103. The data store 127 may be representative of one or more data stores. The data store 127 may include memory of the computing environment 103, persistent storage resources of the computing environment 103, or any other storage resources on which data may be stored by the computing environment 103. In some examples, the data store 127 may include memory of one or more servers. Further, the data store 127 may include one or more relational databases, such as structured query language databases, non-SQL databases, or other relational or non-relational databases.

The data stored in the data store 127 may be associated with the operation of the various applications or functional entities described below. Data stored in the data store 115 may include, for example, patient data 130, dosage plan(s) 133, and machine-readable code(s) 136. The patient data 130 may include various data associated with a patient, such as the patient's medical history, medications that the patient is taking, and data associated with those medications. In some examples, at least a portion of this data may be in the form of an electronic health record (EHR). A dosage plan 133 may include a complete dosage plan or a summary of dosage instructions and other information for a dosing event of one or more medications. A machine-readable code 136 may include a barcode, a QR code, or any other code that encodes data in a manner that can be decoded by a computing device upon being visually scanned by an image capture device. Various applications or other functionality may be executed in the computing environment 103. Components executed in the computing environment 103 may include a natural language processor 139, a summarization module 142, a machine-readable code generator 145, an alert system 148, and other applications, services, processes, systems, engines, or functionality not discussed in detail herein.

The summarization module 142 may be executed to obtain information about one or more medications from various data sources and generate a summary of that information. The summarization module 142 may include a generative artificial intelligence (AI) model like a large language model (LLM).

A summary generated by the summarization module 142 may be provided to the patient device 106 and presented to the patient as a dosage plan 133. The summarization module 142 may obtain data from, for example, the patient device 106, one or more medical provider devices 109, one or more care manager devices 112, and a medication information database 115. Data obtained from any of these sources may be stored in the data store 127 as patient data 127 associated with the patient.

The machine-readable code generator 145 may be executed to generate a machine-readable code based at least in part on a dosage plan 133. A machine-readable code 136 may encode, for example, a textual representation of a dosage plan 133 and any images that may accompany the dosage plan 133, a uniform resource locator (URL) or other identifier that may be used to access the dosage plan 133, or any other data that can enable the patient device 106 to access the dosage plan 133.

The alert system 148 may be executed to provide alerts about the patient's condition and other matters concerning the patient. The alert system 148 may provide alerts in response to being notified of the existence of a condition by, for example, the summarization module 142 or the patient device 106. The alert system 148 may provide alerts to appropriate contacts, including the patient device, one or more medical provider devices 109, or one or more care manager devices 112.

### B. Patient Device

The patient device 106 may represent a client device coupled to the network 124. The patient device 106 may include a processor-based system, such as a computer system, that may include a desktop computer, a laptop computer, a personal digital assistant, a cellular telephone, a smartphone, a set-top box, a music player, a tablet computer system, a game console, an electronic book reader, or any other device with like capability. The patient device 106 may also be equipped with networking capability or networking interfaces, including a localized networking or communication capability, such as a near-field communication (NFC) capability, radio-frequency identification (RFID) read or write capability, or other localized communication capability.

The patient device 106 may include or have access to an image capture device 151. The image capture device 151 may be, for example, a digital camera integrated with the patient device 106. As another example, the image capture device 151 may be a peripheral device such as a digital camera or a scanner coupled to the patient device 106. The image capture device 151 may be operated to process raw input data captured by an image sensor to generate image data. The image data may include a live preview feed generated by the image capture device 151 using the image data or a recorded photo or video generated from the image data.

In addition, the patient device 106 may be configured to execute various applications, such as a chatbot 154 and an augmented reality engine 157. The chatbot 154, the augmented reality engine 157, and potentially other applications may access network content served up by the computing environment 103 or other servers, image data captured by the image capture device 151, and user input provided by a user of the patient device 106 (e.g., the patient) to render an augmented reality interface 160 in a display 163 accessible to the patient device 106. The display 163 may be any display such as a liquid crystal display (LCD), touch-screen display, or other type of display device. The patient device 106 may also be configured to execute a browser or a dedicated application that may render a network page, an application screen, or other interface in the display 163. Further, the patient device 106 may be configured to execute email applications, management applications, enterprise applications, social networking applications, word processors, spreadsheet applications, media viewing applications, or other applications.

The chatbot 154 may be executed to interact with the patient to help ensure care plan adherence. The chatbot 154 may include a generative AI model, such as an LLM. The chatbot 154 may employ various natural language processing techniques to, for instance, synthesize speech from text to provide voice output for a patient, transcribe a patient's speech to text, extract information about a medication from a textual record, or translate information into a patient's preferred language.

The chatbot 154 may notify patients of dosing events and provide visual guidelines through the AR interface 160 to help identify the correct medications. The chatbot 154 may present the patient with a dosing reminder for an upcoming dosing event.

The chatbot 154 can recognize medications, containers, and organizers using object recognition and optical character recognition (OCR) to ensure the correct medication is taken. The chatbot 154 may present dosage plans 133 and prompt the patient to ensure that medication conditions for taking medications have been met. If an incorrect medication is identified, the chatbot 154 may notify the alert system 148, which may alert one or more medical provider devices 109 and or care manager devices 112. Additionally, the chatbot 154 may answer patient questions about the dosing event and monitor the patient's condition after medication is taken, notifying the alert system 148 if adverse conditions are detected, which may then alert one or more medical provider devices 109 and/or care manager devices 112.

The chat bot 154 may identify a medication based on a dose of the medication, a medication label, or a compartment of a medication organizer from image data captured by the image capture device 151. The chat bot 154 may identify the medication by performing feature extraction to isolate the visual features of the medication within the image data, matching the extracted features to an image of the medication, and recognizing the medication object within the image data based on the matched features. In some implementations, the chat bot 154 may engage low-light image capture features of the image capture device 151 to enable the chat bot 154 to identify the medication (and other objects discussed below) even in low light environments.

The chatbot 154 may present information and interact with the patient in different ways depending on the preferences or needs of the patient. For instance, the chatbot 154 may present information using text, images, or voice. When presenting information using text, the chatbot 154 may present, in the AR interface 160, the information in a large font if the patient has issues with eyesight. The chatbot 154 may likewise cause the AR interface 160 to present images, such as images of a medication, at a larger scale if the patient has eyesight issues. If a patient prefers or would not be capable of easily reading text, the chatbot 154 may present information via voice using text-to-speech synthesis in a language that the patient speaks or using a type of voice that the patient prefers.

The chatbot 154 may generate and render the augmented reality interface 160 in the display 163. The chat bot 154 may generate the AR interface 160 using image data captured by the image capture device and various data from the chatbot 154. The AR interface 160 may help a patient identify a medication that needs to be taken. For example, the chat bot 154 may present, in the AR interface 160, annotations, labels, callouts, and other visual elements that help the patient to identify medication. For example, the chat bot 154 may present an image of the medication or a physical description of the medication in the AR interface 160. As another example, the chat bot 154 may, within the AR interface 160, highlight or otherwise emphasize the correct medication, a container of the correct medication, or a medication organizer compartment that includes the correct medication.

As another example, the chatbot 154 may cause the AR interface 160 to present visual guidelines or other prompts that help a patient follow an instruction. To illustrate, when the patient is attempting to take a medication, the chatbot 154 may, within the AR interface 160, highlight or otherwise visually emphasize a compartment of a medication organizer that contains the correct medication. To further illustrate, when presenting a dosage plan 133 at the time of a dosing event, the chatbot 154 may cause the AR interface 160 to present a checklist that includes each aspect of the dosage plan 133 as an item in the checklist.

### C. Medical Provider Devices

The medical provider device(s) 109 may represent one or more medical provider devices 109 coupled to the network 124. The medical provider device 109 may include a processor-based system, such as a computer system, that may include a desktop computer, a laptop computer, a personal digital assistant, a cellular telephone, a smartphone, a set-top box, a music player, a tablet computer system, a game console, an electronic book reader, or any other device with like capability. The medical provider device 109 may also be equipped with networking capability or networking interfaces, including a localized networking or communication capability, such as a near-field communication (NFC) capability, radio-frequency identification (RFID) read or write capability, or other localized communication capability.

A medical provider device 109 may be associated with a medical provider of the patient. The medical provider device 109 may transmit medical and other data to the summarization module 142, patient device 106, or care manager device 112.

### D. Care Manager Device

The care manager device(s) 112 may represent one or more care manager devices 112 coupled to the network 124. The care manager device 112 may include a processor-based system, such as a computer system, that may include a desktop computer, a laptop computer, a personal digital assistant, a cellular telephone, a smartphone, a set-top box, a music player, a tablet computer system, a game console, an electronic book reader, or any other device with like capability. The care manager device 112 may also be equipped with networking capability or networking interfaces, including a localized networking or communication capability, such as a near-field communication (NFC) capability, radio-frequency identification (RFID) read or write capability, or other localized communication capability.

The care manager device 112 may include or have access to an image capture device. The care manager's image capture device may be, for example, a digital camera integrated with the care manager device. As another example, the care manager's image capture device may be a peripheral device such as a digital camera or a scanner coupled to the care manager device 112. The care manager's image capture device may be operated to process raw input data captured by an image sensor to generate image data. The image data may include a live preview feed generated by the care manager's image capture device using the image data or a recorded photo or video generated from the image data.

The care manager device 112 may be associated with a family member of the patient, a home health provider, a case manager, or other caregiver for the patient. The care manager device 112 may operate a image capture device to capture image data representing a medication prescribed to the patient and any records associated with the medication. The care manager device 112 may provide this data to the summarization module 142. In some implementations, the care manager device 112 may extract text data from the image data and potentially determine, using the text data, an identity of the medication, a quantity of available doses of the medication, how often the doses should be administered (i.e., the dosing interval), any other instructions included in the record, and any other pertinent information included in the record. This data may be provided to the summarization module 142 instead of or in addition to the image data.

### E. Medication Information Database

The medication information database 115 may represent one or more data stores. The medication information database 115 may be implemented in one or more computing devices, such as one or more server devices. The medical information database 115 may include one or more relational databases, such as structured query language databases, non-SQL databases, or other relational or non-relational databases.

The medical information database 115 may be a database of publicly available information about various different medications. This information may include, for instance, drug interaction information, possible side effects, or ingredients. The medical information database 115 may be maintained by, for example, a governmental entity, a pharmaceutical entity, or a medical provider.

### III. Identifying a New Medication

When the patient receives a new or additional medication, a care manager device 112 (or patient device 106 or medical provider device 109) may cause the care manager's image capture device to capture image data representing a label on the medication's container, a prescription for the medication, instructions for taking the medication, or any other record associated with the medication. The care manager device 112 (or patient device 106 or medical provider device 109) may cause the care manager's image capture device to capture image data representing a single dose of the medication. The care manager device 112 may provide this image data to the summarization module 142.

The summarization module 142 may then determine, using text data extracted from image data, an identity of the medication, a quantity of available doses of the medication, how often the doses should be administered (i.e., the dosing interval), any other instructions included in the record, and any other pertinent information included in the record. The summarization module 142 may store this information in the patient data 130.

### IV. Generating Dosage Plan

The summarization module 142 may generate dosage plans 133 based on information obtained from various sources, some of which may be stored in and accessed from the patient data 130. The summarization module 142 may obtain data from, for example, the patient device 106, one or more medical provider devices 109, one or more care manager devices 112, and a medication information database 115. The summarization module 142 may generate one or more dosage plan 133 when the summarization module 142 receives information about a new medication in the form of image data or other data about a new medication from a care manager device 112 (or a medical provider device 109 or patient device 106).

From one or more medical provider devices 109, the summarization module 142 may obtain medical data associated with the patient. The summarization module 142 may obtain medical data from potentially multiple different medical provider devices 109, each being associated with a different medical provider. For example, if a patient has been prescribed a first medication by a cardiologist and a second medication by a dermatologist, the summarization module 142 may obtain at least dosing instructions for the first medication from a medical provider device 109 associated with the cardiologist and dosing instructions for the second medication from a medical provider device 109 associated with the dermatologist, as well as potentially obtaining the patient's medical history from a medical provider device 109 associated with the patient's primary care provider.

The summarization module 142 may obtain dosage instructions from a medical provider device 109. Dosage instructions for a medication may include information such as the quantity of the medication that should be taken in each dose, the medication's dosing interval, any food or liquid directives for taking the medication, and any activity directives for taking the medication. The summarization module 142 may also obtain medical information associated with the patient from one or more medical provider devices 109, such as the patient's medical history, any over-the-counter medications that the patient is taking, information about any allergies that the patient may have, and information about any other medical conditions that the patient may have.

From one or more care manager devices 112, the summarization module 142 may obtain one or more images of one or more medications. The image of a medication may be received from a care manager device 112 when the patient receives the medication.

From the medication information database 115, the summarization module 142 may obtain publicly-available information about one or more medications. This information may include, for instance, drug interaction information, possible side effects, and/or ingredients.

Once the summarization module 142 has obtained information from the various sources as discussed above, the summarization module 142 may generate one or more dosage plan(s) 133 based on this information. The summarization module 142 may determine what time(s) during in recurring period of time that the patient should take one or more medications based on the dosing interval for the one or more medications. The recurring period may be, for example, a twenty-four-hour period. If the one or more medications comprise a plurality of medications, the summarization module 142 may correlate the time(s) at which each medication should be taken. The summarization module 142 may identify each instance that one or more medications should be taken during the recurring period as a single dosing event. Each dosing event may include taking one or multiple medications.

The summarization module 142 may generate a dosage plan 133 for each dosing event identified within the recurring period. The dosage plan 133 for a dosing event may include, for example, what medication(s) should be taken in that dosing event, the dosage instructions for the one or more medications of the dosing event, and an image of each of the one or more medications. The dosage plan 133 may combine and summarize the dosage instructions for multiple medications into a single set of cohesive instructions that concisely instructs the patient on what medications to take, how each medication should be taken, any and all food or liquid directives the patient should follow, and any and all activity directives that the patient should follow.

When attempting to generate a dosage plan 133, the summarization module 142 may detect any conflicts between the dosage instructions for each medication and the dosage instructions for any other medications the patient is taking, the patient's medical history, the patient's allergies, or any other medical condition the patient has. For example, the summarization module 142 may detect that the patient is allergic to a medication, that a medical condition of the patient is a contraindication for taking a medication, or that a harmful interaction exists between a medication and another medication that the patient is taking. As another example, the summarization module 142 may detect that an allergy or medical condition of the patient contraindicates a food or liquid directive from the dosage instructions for a medication, such as an allergy to a food, a medical condition that renders harmful a directive to avoid eating food for a certain duration before or after taking a medication, or that the dosage instructions for one medication direct a patient to avoid ingesting food for a certain duration before or after taking the medication while the dosage instructions for another medication would direct the patient to ingest food within that certain duration. To give an additional example, the summarization module may detect that the dosage instructions for a medication direct the patient to perform an activity (e.g., stretches) that the patient's medical history indicates that the patient is unable to or should not perform or that the dosage instructions for one medication direct the patient to perform a particular activity while the dosage instructions for another medication direct the patient to avoid performing the particular activity.

If the summarization module 142 detects any such conflict, the summarization module may notify the alert system 148, which may transmit an alert to one or more medical provider devices 109 and/or one or more care manager devices 112. The summarization module 142 may also refrain from generating a dosage plan 133.

### V. Generating Machine-Readable Codes

The machine-readable code generator 145 may generate a machine-readable code 136 for a particular dosage plan 133 such that the patient device 106 may access the particular dosage plan 133 by scanning that machine-readable code 136. For example, the machine-readable code generator 145 may encode a textual representation of a particular dosage plan 133 and any images that may accompany the dosage plan 133. As another example, the machine-readable code generator 145 may encode a uniform resource locator (URL) or other identifier that may be used to access a particular dosage plan 133. After generating a machine-readable code 145, the machine-readable code generator 145 may provide the machine-readable code 136 to the patient device 106, a medical provider device 109, or a care manager device 112. The machine-readable code 136 may then be, for example, printed using a printing device and affixed to the patient's medication organizer.

### VI. Completing Dosing Events

The summarization module 142 may detect, based on the patient data 130 and/or dosage plan 133, when it is time for the patient to complete a dosing event. The summarization module 142 may transmit a notification about the dosing event, as well as an identification of one or more medications to be taken during the dosing event. In some implementations, the summarization module 142 may also transmit a dosage plan 133 corresponding to that dosage event to the chatbot 154, as well as potentially transmitting information about and/or image data of one or more medications to be taken during the dosing event and other data associated with the patient from the patient data 130. In other implementations, however, the chatbot 154 may fetch some or all of this data only when needed, or not at all.

The chatbot 154 may likewise notify the patient that it is time to complete a dosing event. For example, the chatbot 154 may send a push notification or display a prompt or dialog box in the display 163 of the patient device 106 notifying the patient that it is time to take medication.

The chatbot 154 may cause the AR interface 160 to present one or more visual guidelines to help the patient identify which medication(s) that the patient should take. For example, the AR interface 160 may present the name, a physical description, and an image of the medication. As another example, the chatbot 154 may instruct the patient to one or more medications, one or more medication containers, or a medication organizer in a frame of the image capture device 151 so that the chatbot 154 may determine which medication(s) are correct medications for the current dosing event based on medication information received from the summarization module 142 with reference to the current dosing event.

### A. Identifying Medications

The chatbot 154 may identify a medication and validate whether the medication, medication container, or medication organizer compartment included in captured image data corresponds to a correct medication. In some implementations, the chatbot 154 may present one or more visual guidelines or otherwise prompt the patient to position one or more medications, one or more medication containers, or a medication organizer within a frame of the image capture device 151 to help the patient identify which medications to take as part of the dosing event.

For example, the patient may position a medication within a frame of the image capture device 151. The chat bot 154 may perform object recognition on the medication from the image data as described above. In some implementations, the chatbot 154 may determine whether the recognized medication matches image data for the correct medication received from the summarization module 142. In other implementations, the chatbot 154 may provide data identifying the recognized medication to the summarization module 142, which itself may determine whether the recognized medication matches image data for the correct medication based on the patient data 130 and notify the chat bot 154 of the result.

As another example, the patient may position a medication container within a frame of the image capture device 151. The chat bot 154 may perform optical character recognition on the medication container label from the image data to extract the corresponding text data. In some implementations, the chat bot 154 may compare the extracted text data with information about the correct medication received from the summarization module 142 to determine whether it matches the correct medication. In other implementations, the chatbot 154 may provide the extracted text data (or the image data, without extracting text data) to the summarization module 142, which itself may determine whether text data extracted from the image data matches data from the medication container label of the correct medication based on the patient data 130 and notify the chat bot 154 of the result.

As an additional example, the patient may position a medication organizer within the frame of the image capture device 151. The chat bot 154 may identify, from the image data, one or more machine-readable codes 136 affixed to an outer surface of the medication organizer. The chat bot 154 may decode the machine-readable code 136 to access a dosage plan 133. The chat bot 154 may determine whether the dosage plan 133 includes instructions to take the correct medicine.

If an identified medication, medication container, or machine-readable code 136 matches a correct medication, then the chatbot 154 may proceed with presenting the dosage plan 133 for that medication in the AR interface 160. In some examples, though, the chatbot 154 may present further visual guidelines or prompts to help the patient identify additional medications from the dosing event and repeat the process discussed above

If an identified medication is not a correct medication, then the chatbot 154 may cause the AR interface 160 to present a warning or other notification that the identified medication should not be taken at the current time. In some implementations, the chatbot 154 may notify the alert system 148 that the chatbot 154 has identified an incorrect medication, and the alert system 148 may transmit an alert to one or more care provider devices 112 and/or one or more medical provider devices 109.

### B. Presenting Dosage Plans

The chat bot 154 may cause the AR interface 160 to present a dosage plan 133 for dosing event. The chat bot 154 may cause the AR interface 160 to present dosage plan 133 in response to, for example, medication identification module 155 identifying the medication, the medication container and/or container label, or a machine-readable code 136 corresponding to the medication in image data captured by the image capture device 151. For example, the dosage plan 133 presented in the AR interface 160 may include an identification of each correct medication (including image(s) and physical description of each correct medication), instructions for administering each correct medication, the dosage condition for each correct medication, and instructions for the patient after taking each correct medication (e.g., refraining from driving).

If the chat bot 154 identifies an incorrect medication, a container of an incorrect medication, or a medication organizer compartment of an incorrect medication, the chat bot 154 may refrain from presenting dosage plans 133 for the incorrect medication in the AR interface 160. The chat bot 154 may also present, in the AR interface 160, an indication that the incorrect medication is not part of a current dosing event. In some implementations, the chat bot 154 may advise the patient that the time since the patient last took the incorrect medication is less than the incorrect medication's dosing interval (i.e., the time interval between doses of a medication) and/or present a reminder of what time the patient is next directed to take that medication.

The AR interface 160 may help a patient to correctly dose one or more medications allocated to a particular compartment of a medication organizer. When, for example, the compartment is positioned within the frame of the image capture device, the chat bot 154 may cause the AR interface 160 to present dosage plan 133 for and/or identifications of every medication allocated to that compartment. The chat bot 154 may also identify individual medications that are actually present within the compartment. If the chatbot 154 fails to identify, within that compartment, any medication allocated to that compartment, the chat bot 154 may cause the AR interface 160 to present an identification of the missing medication. Likewise, if the chatbot 154 identifies, within the compartment, any medication that is not allocated to that compartment, the chat bot 154 may present, in the AR interface 160, an indication that the patient is not directed to take the identified medication in a same dosing event as other medication(s) allocated to that compartment. The chat bot 154 may, if applicable, also present, in the AR interface 160, dosage plan 133 for the identified medication. In addition, the chat bot 154 may highlight or otherwise emphasize, within the AR interface 160, a compartment of the medication organizer to which the identified medication is allocated.

### C. Ensuring Completion of Dosing Events

When the patient is attempting to complete a dosing event, the chatbot 154 may enable the patient to ask questions about various aspects of the dosing event, medications, or other matters. For example, the patient may ask for clarification of instructions for taking a medication as given by a dosage plan 133, about whether a particular medication is a correct medication, or about food or liquid directives or activity directives that accompany a particular medication. The chatbot 154 may process the patient's question and provide an appropriate response.

The chatbot 154 may present a prompt that inquires whether the patient has met the dosage condition(s) for a current dosing event. The chatbot 154 may then receive user input from the patient in response to the prompt; if the patient's response indicates that the patient has not met a dosage condition, the chatbot 154 may take corrective action. To illustrate, if the care plan directs the patient to take a medication in a current dosing event just after eating food, the chatbot 154 may ask the patient whether the patient has already eaten food. If the patient provides a negative response, the chatbot 154 may provide a response that advises the patient to eat food before completing the current dosing event. The chatbot 154 may repeat this interaction periodically until the patient indicates that the dosage condition has been met.

After the chat bot 154 has prompted the patient to complete a dosing event, the chatbot 154 may present a prompt requesting confirmation that the patient has, in fact, completed the dosing event by taking the correct medications. If the patient's response indicates that the patient did not actually take a medication, the chatbot 154 may provide a response that advises the patient to take the medication. The chatbot 154 may repeat this interaction periodically until the patient indicates that the patient did take the medication. If the patient does not confirm completion of the dosing event within a predetermined period of time, the chatbot 154 may notify the alert system 148, which may alert a medical provider device 109 and/or a care manager device 112.

The chatbot 154 may present a prompt about the patient's condition. This prompt may be presented when a predetermined amount of time has elapsed since a most recent dosing event was completed. The chatbot 154 may analyze the patient's response and determine whether the response could be interpreted as meaning that the patient's current condition is adverse. If so, the chatbot may notify the alert system 148, which may provide an alert to a medical provider device 109 and/or a care manager device 112.

### VII. Examples of Augmented Reality Interface

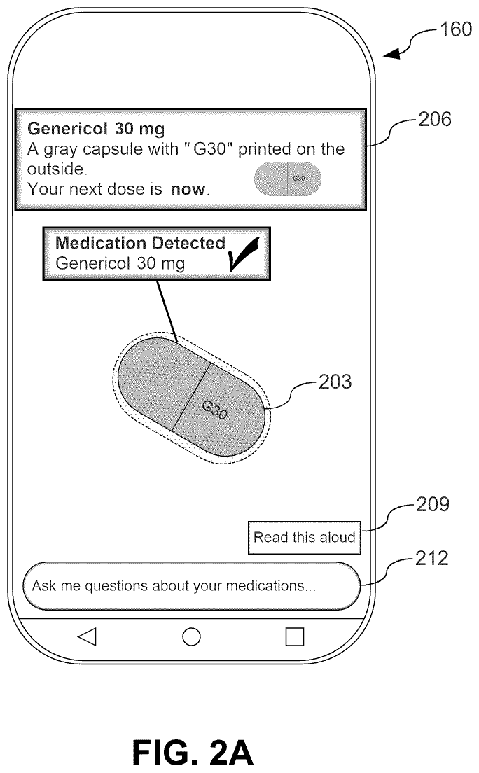

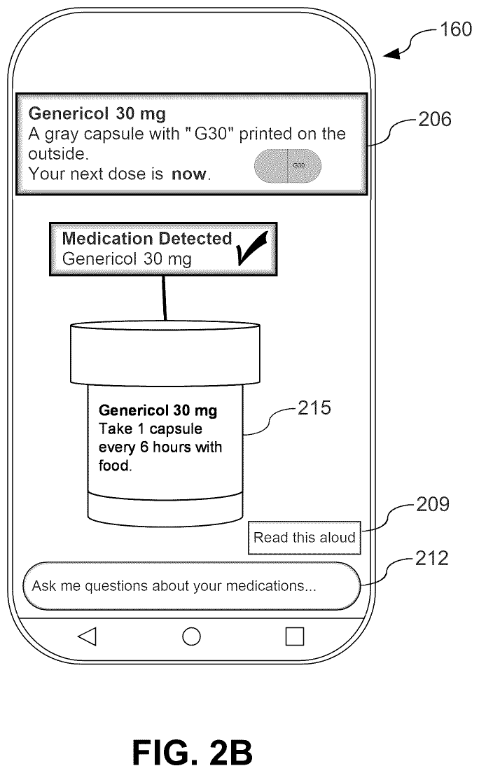

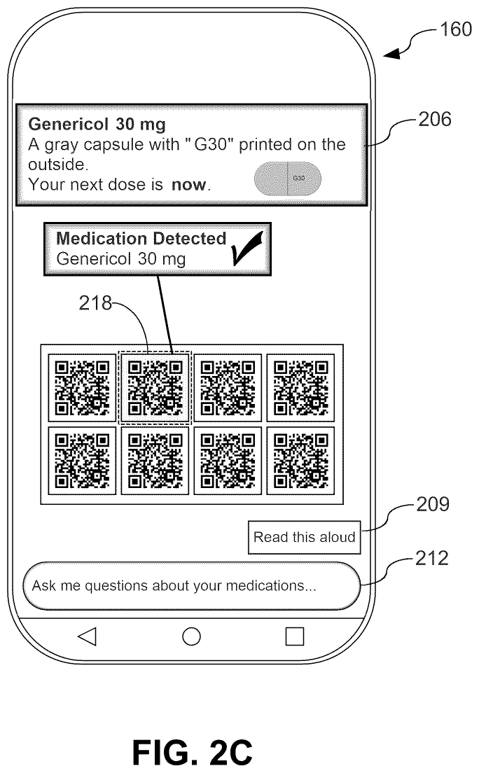

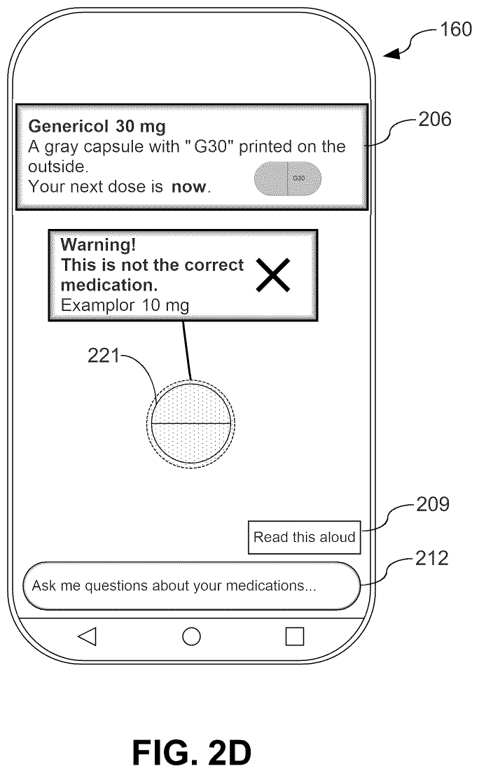

FIGS. 2A-2D, 3, and 4A-4B are diagrams depicting various examples of the AR interface 160 rendered in the display 163 of the patient device 106. The examples of the AR interface 160 depicted in FIGS. 2A-2D, 3, and 4A-4B may be related to functionality described above with respect to FIG. 1. The examples of the AR interface 160 depicted in the FIGS. 2A-2D, 3, and 4A-4B are simply illustrative examples, however, and the functionality described may be implemented using any suitable user interface.

The examples of the AR interface 160 in FIGS. 2A-2D, 3, and 4A-4B may be presented to facilitate the patient completing a dosing event. In these examples, the dosing event includes taking a 30 mg dose of a medication called “Genericol”.

### A. Augmented Reality Interface Used to Identify Medications

FIGS. 2A-2D depict examples in which the AR interface 160 is used to identify a medication related to a dosing event, in various embodiments.

FIG. 2A shows an example of the AR interface 160 being used to identify a medication as a correct medication based on image data depicting a single dose of the medication. In FIG. 2A, the image capture device 151 has captured image data representing a medication capsule 203 and rendered the image data in the AR interface 160. The AR interface 160 has highlighted the medication capsule 203 within the rendering of the image data and identified the capsule 203 as the correct medication, i.e., a 30 mg dose of “Genericol”. The chatbot 154 has identified the medication capsule 203 as the correct medication using object recognition, which may include comparing features of the medication capsule 203 extracted from the image data with an image of the correct medication from the patient data 130.

The AR interface 160 includes a message box 206 that advises the patient that the patient is due to take a 30 mg dose of the medication “Genericol” and includes a physical description and image of “Genericol”. The information about and image of “Genericol” may be obtained from the summarization module 142. The AR interface 160 further includes a selectable component 209 that, if selected, would cause the chatbot 154 to output speech synthesized at least from the text presented in the AR interface 160. The AR interface 160 further includes an input box 212 that enables the patient to input text to query the chatbot 154.

FIG. 2B shows an example of the AR interface 160 being used to identify a medication as a correct medication based on a medication container. In FIG. 2B, the image capture device 151 has captured image data representing a medication container 215 and rendered the image data in the AR interface 160. The AR interface 160 has highlighted the medication container 215 within the rendering of the image data and identified the medication container 215 as corresponding to the correct medication, i.e., a 30 mg dose of “Genericol”. The chatbot 154 has identified the medication container 215 as containing the correct medication by extracting text from a label of the medication container 215 and matching the text with text of the label from the patient data 130. The AR interface includes the message box 206, selectable component 209, and input box 212 as discussed with reference to FIG. 2A.

FIG. 2C shows an example of the AR interface 160 being used to identify a medication as a correct medication based on a machine-readable code 136. In FIG. 2C, the image capture device 151 has captured image data representing a medication organizer and rendered the image data in the AR interface 160. The AR interface 160 has highlighted a compartment 218 of the medication organizer 218 corresponding to the correct medication within the rendering of the image data. The AR interface 160 has identified the compartment 218 of the medication organizer as corresponding to the correct medication, i.e., a 30 mg dose of “Genericol”. The chatbot 154 has identified the compartment 218 of the medication organizer as containing the correct medication by decoding a machine-readable code 136 affixed to an outer surface of the compartment 218. The chatbot 154 has decoded the machine-readable code 136 to access a dosage plan 133 and determined that the dosage plan 133 specifies the correct medication. The AR interface includes the message box 206, selectable component 209, and input box 212 as discussed with reference to FIG. 2A.

FIG. 2D shows an example of the AR interface 160 being used to identify a medication as an incorrect medication based on image data depicting a single dose of the medication. In FIG. 2D, the image capture device 151 has captured image data representing a medication tablet 221 and rendered the image data in the AR interface 160. The AR interface 160 has highlighted the medication tablet 221 within the rendering of the image data and identified the medication tablet 221 as an incorrect medication, i.e., a 50 mg dose of the medication “Examplor” rather than a 30 mg dose of “Genericol”. The chatbot 154 has identified the medication tablet 221 as the incorrect medication using object recognition, which may include comparing features of the medication tablet 221 extracted from the image data with an image of the correct medication from the patient data 130. The AR interface includes the message box 206, selectable component 209, and input box 212 as discussed with reference to FIG. 2A.

### B. Augmented Reality Interface to Present Dosage Plans

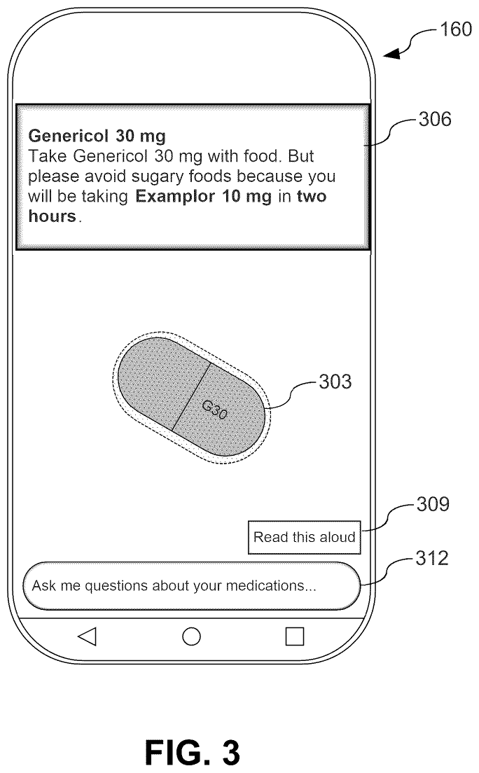

FIG. 3 shows an example in which the AR interface 160 is used to present a dosage plan 133, in an embodiment. In this example, the dosing event includes taking a 30 mg dose of a medication called “Genericol”.

As in FIG. 2A, AR interface 160 has highlighted a medication capsule 303 identified as the correct medication “Genericol”. The AR interface 160 includes a message box 306 that comprises a dosage plan 133 for a current dosing event. The AR interface 160 further includes a selectable component 309 and input box 312 similar to those discussed with reference to FIG. 2A.

### C. Augmented Reality Interface to Ensure Completion of Dosing Event

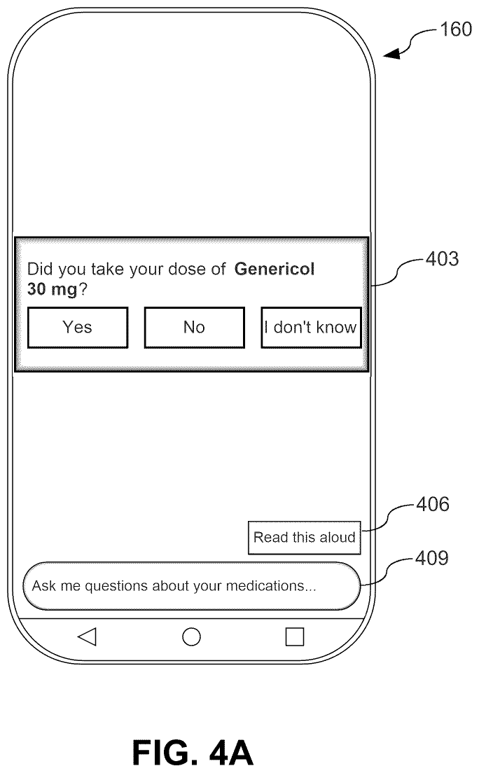

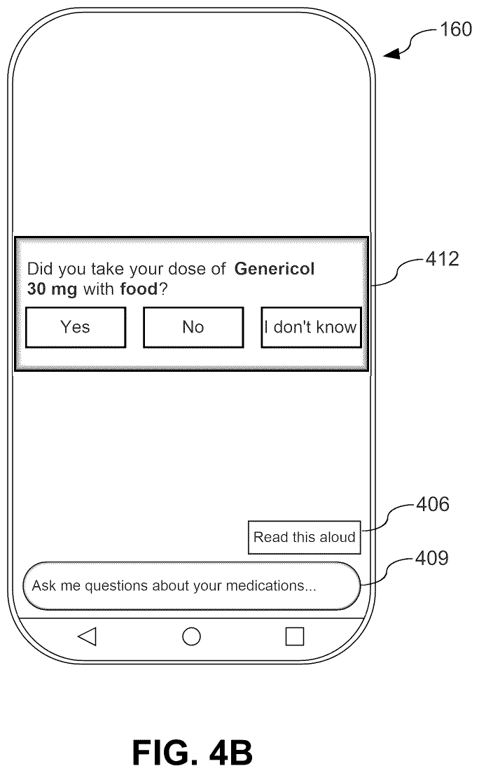

FIGS. 4A-4B depict examples in which the AR interface 160 is used to prompt the patient to ensure completion of a current dosing event, in various embodiments. In these examples, the dosing event includes taking a 30 mg dose of a medication called “Genericol”.

FIG. 4A shows an example of the AR interface 160 being used to prompt the patient to confirm that the patient has taken the medication “Genericol”. The AR interface 160 includes a dialog box 403 that asks the patient whether the patient took the 30 mg dose of the medication “Genericol”. The dialog box 403 may be presented after a predetermined period of time has elapsed after presenting the dosage plan 133 as in FIG. 3. The dialog box 403 allows a patient to select among three options: one to confirm that the patient has taken the medication, one to deny that the patient has taken the medication, and one to indicate that it is unknown whether the patient has taken the medication. If “No” is selected, the AR interface 160 may again prompt the user to take the medication, which may again include identifying the medication and/or presenting the dosage plans 133 as described with respect to FIGS. 2A-2D and FIG. 3. The AR interface 160 may then present the dialog box 403 and again prompt the patient to confirm that the patient has taken the medication. If this recurs a predetermined number of times, the chatbot 154 may notify the alert system 148, which may provide an alert to one or more medical provider devices 109 and/or one or more care manager devices 112. If “I don't know” is selected, then the chatbot 154 may notify the alert system 148, which may provide an alert to one or more medical provider devices 109 and/or one or more care manager devices 112. The AR interface 160 further includes a selectable component 406 and input box 409 similar to those discussed with reference to FIG. 2A.

FIG. 4B shows an example of the AR interface 160 being used to prompt the patient to confirm that the patient has met a dosage condition for a dosing event. The dosage condition in this example is a food directive. The AR interface 160 includes a dialog box 412 that asks the patient whether the patient took the 30 mg dose of the medication “Genericol” with food. This dialog box 412 may be presented after the patient confirms that the patient has taken the 30 mg dose of the medication “Genericol”. The dialog box 403 allows a patient to select among three options: one to confirm that the patient did take the medication with food, one to deny that the patient took the medication with food, and one to indicate that it is unknown whether the patient took the medication with food. If “No” is selected, the AR interface 160 may prompt the user to ingest food, which may again include displaying a portion of the dosage plan 133 that relates to the food directive for the medication “Genericol”. The AR interface 160 may then present the dialog box 412 and again prompt the patient to confirm that the patient has taken the medication after a predetermined period of time has elapsed. If this recurs a predetermined number of times, the chatbot 154 may notify the alert system 148, which may provide an alert to one or more medical provider devices 109 and/or one or more care manager devices 112. If “I don't know” is selected, then the chatbot 154 may notify the alert system 148, which may provide an alert to one or more medical provider devices 109 and/or one or more care manager devices 112. The AR interface 160 includes the selectable component 406 and input box 409 discussed with reference to FIG. 4A.

### Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

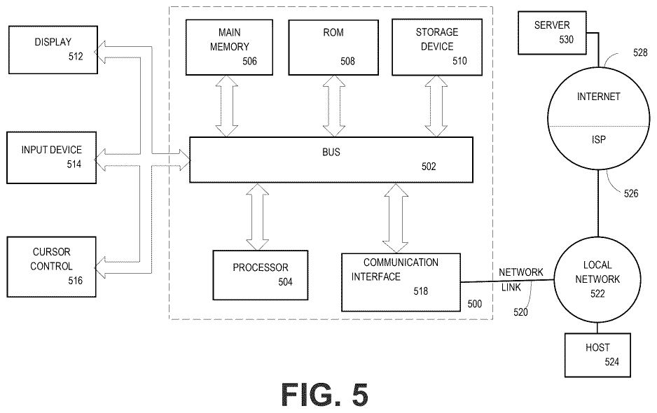

For example, FIG. 5 is a block diagram that illustrates a computer system 500 upon which an embodiment of the invention may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk, optical disk, or solid-state drive is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage device 510. Volatile media includes dynamic memory, such as main memory 506. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid-state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### Software Overview [Boilerplate]

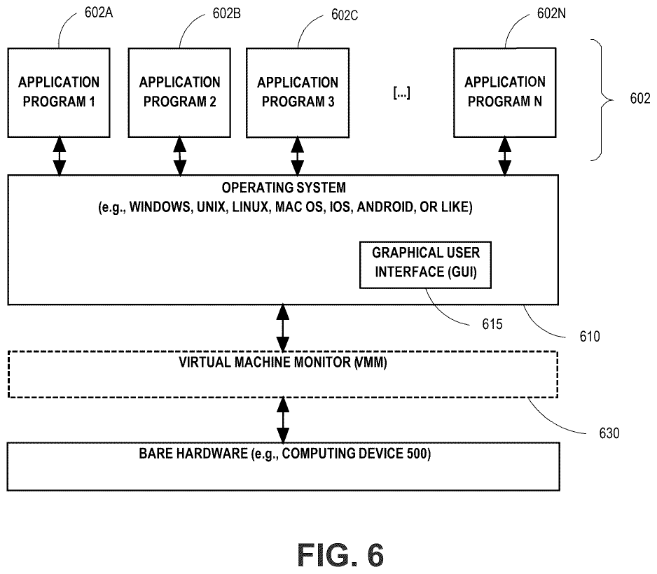

FIG. 6 is a block diagram of a basic software system 600 that may be employed for controlling the operation of computing system 500. Software system 600 and its components, including their connections, relationships, and functions, is meant to be exemplary only, and not meant to limit implementations of the example embodiment(s). Other software systems suitable for implementing the example embodiment(s) may have different components, including components with different connections, relationships, and functions.

Software system 600 is provided for directing the operation of computing system 500. Software system 600, which may be stored in system memory (RAM) 506 and on fixed storage (e.g., hard disk or flash memory) 510, includes a kernel or operating system (OS) 610.

The OS 610 manages low-level aspects of computer operation, including managing execution of processes, memory allocation, file input and output (I/O), and device I/O. One or more application programs, represented as 602A, 602B, 602C . . . 602N, may be “loaded” (e.g., transferred from fixed storage 510 into memory 506) for execution by the system 600. The applications or other software intended for use on computer system 500 may also be stored as a set of downloadable computer-executable instructions, for example, for downloading and installation from an Internet location (e.g., a Web server, an app store, or other online service).

Software system 600 includes a graphical user interface (GUI) 615, for receiving user commands and data in a graphical (e.g., “point-and-click” or “touch gesture”) fashion. These inputs, in turn, may be acted upon by the system 600 in accordance with instructions from operating system 610 and/or application(s) 602. The GUI 615 also serves to display the results of operation from the OS 610 and application(s) 602, whereupon the user may supply additional inputs or terminate the session (e.g., log off).

OS 610 can execute directly on the bare hardware 620 (e.g., processor(s) 504) of computer system 500. Alternatively, a hypervisor or virtual machine monitor (VMM) 630 may be interposed between the bare hardware 620 and the OS 610. In this configuration, VMM 630 acts as a software “cushion” or virtualization layer between the OS 610 and the bare hardware 620 of the computer system 500.

VMM 630 instantiates and runs one or more virtual machine instances (“guest machines”). Each guest machine comprises a “guest” operating system, such as OS 610, and one or more applications, such as application(s) 602, designed to execute on the guest operating system. The VMM 630 presents the guest operating systems with a virtual operating platform and manages the execution of the guest operating systems.

In some instances, the VMM 630 may allow a guest operating system to run as if it is running on the bare hardware 620 of computer system 600 directly. In these instances, the same version of the guest operating system configured to execute on the bare hardware 620 directly may also execute on VMM 630 without modification or reconfiguration. In other words, VMM 630 may provide full hardware and CPU virtualization to a guest operating system in some instances.

In other instances, a guest operating system may be specially designed or configured to execute on VMM 630 for efficiency. In these instances, the guest operating system is “aware” that it executes on a virtual machine monitor. In other words, VMM 630 may provide para-virtualization to a guest operating system in some instances.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A method comprising:
receiving, by one or more computing devices, information associated with one or more medications from a public database;
receiving, by the one or more computing devices, an image of the one or more medications;
receiving, by the one or more computing devices, dosage instructions for the one or more medications from one or more medical provider devices;
generating, by the one or more computing devices, a dosage plan based at least in part on the information associated with the one or more medications, the image of the one or more medications, and the dosage instructions;
generating, by the one or more computing devices, a machine-readable code for accessing the dosage plan; and
providing, by the one or more computing devices, the machine-readable code to a client device.

2. The method of claim 1, wherein the one or more medications comprise a first medication, the dosage instructions comprise first dosage instructions, the one or more medical provider devices comprise a first medical provider device, and the method further comprising:
receiving, by the one or more computing devices, second dosage instructions for a second medication from a second medical provider device;
identifying, by the one or more computing devices, one or more conflicts between the first dosage instructions and the second dosage instructions; and
generating, by the one or more computing devices, the dosage plan further based on the second dosage instructions and the one or more conflicts.

3. The method of claim 1, further comprising generating, by the client device, an augmented reality (AR) overlay over imagery captured by an image capture device, the AR overlay comprising the image of the one or more medications, at least a portion of the dosage plan, one or more questions, and one or more reminders.

4. The method of claim 3, further comprising generating, by the client device, text-to-speech output of the at least the portion of the dosage plan, the one or more questions, and the one or more reminders.

5. The method of claim 3, further comprising:
identifying, by the client device, the one or more medications within the imagery captured by the image capture device; and
modifying, by the image capture device, the AR overlay to visually distinguish the one or more medications.

6. The method of claim 1, further comprising providing, by the one or more computing devices, an alert to the one or more medical provider devices in response to detecting a missed dosage of the one or more medications.

7. The method of claim 1, further comprising providing, by the one or more computing devices, a dosing reminder to the client device.

8. One or more non-transitory storage media storing instructions which, when executed by one or more computing devices, cause:
receiving information associated with one or more medications from a public database;
receiving an image of the one or more medications;
receiving dosage instructions for the one or more medications from one or more medical provider devices;
generating a dosage plan based at least in part on the information associated with the one or more medications, the image of the one or more medications, and the dosage instructions;
generating a machine-readable code for accessing the dosage plan; and
providing the machine-readable code to a client device.

9. The one or more non-transitory storage media of claim 8, wherein the one or more medications comprise a first medication, the dosage instructions comprise first dosage instructions, the one or more medical provider devices comprise a first medical provider device, and the instructions, when executed by the one or more computing devices, further cause:
receiving second dosage instructions for a second medication from a second medical provider device;
identifying one or more conflicts between the first dosage instructions and the second dosage instructions; and
generating the dosage plan further based on the second dosage instructions and the one or more conflicts.

10. The one or more non-transitory storage media of claim 8, further comprising generating, by the client device, an augmented reality (AR) overlay over imagery captured by an image capture device, the AR overlay comprising the image of the one or more medications, at least a portion of the dosage plan, one or more questions, and one or more reminders.

11. The one or more non-transitory storage media of claim 10, wherein the client devices generates text-to-speech output of the at least the portion of the dosage plan, the one or more questions, and the one or more reminders.

12. The one or more non-transitory storage media of claim 10, wherein the client device identifies the one or more medications within the imagery captured by the image capture device and modifies the AR overlay to visually distinguish the one or more medications.

13. The one or more non-transitory storage media of claim 8, further comprising providing, by the one or more computing devices, an alert to the one or more medical provider devices in response to detecting a missed dosage of the one or more medications.

14. The one or more non-transitory storage media of claim 8, further comprising providing, by the one or more computing devices, a dosing reminder to the client device.

15. An apparatus, comprising:
one or more processors; and
one or more memories storing instructions that, when executed by the one or more processors, cause:
receiving information associated with one or more medications from a public database;
receiving an image of the one or more medications;
receiving dosage instructions for the one or more medications from one or more medical provider devices;
generating a dosage plan based at least in part on the information associated with the one or more medications, image of the one or more medications, and instructions;
generating a machine-readable code for accessing the dosage plan; and
providing the machine-readable code to a client device.

16. The apparatus of claim 15, wherein the one or more medications comprise a first medication, the dosage instructions comprise first dosage instructions, the one or more medical provider devices comprise a first medical provider device, and the instructions, when executed by the one or more computing devices, further cause:
receiving second dosage instructions for a second medication from a second medical provider device;
identifying one or more conflicts between the first dosage instructions and the second dosage instructions; and
generating the dosage plan further based on the second dosage instructions and the one or more conflicts.

17. The apparatus of claim 15, further comprising generating, by the client device, an augmented reality (AR) overlay over imagery captured by an image capture device, the AR overlay comprising the image of the one or more medications, at least a portion of the dosage plan, one or more questions, and one or more reminders.

18. The apparatus of claim 17, wherein the client devices generates text-to-speech output of the at least the portion of the dosage plan, the one or more questions, and the one or more reminders.

19. The apparatus of claim 17, wherein the client device identifies the one or more medications within the imagery captured by the image capture device and modifies the AR overlay to visually distinguish the one or more medications.

20. The apparatus of claim 15, further comprising providing, by the one or more computing devices, an alert to the one or more medical provider devices in response to detecting a missed dosage of the one or more medications.
